(19) United States
(12) Patent Application Publication
(10) Pub. No.: US 2020/0392351 A1
(43) Pub. Date: Dec. 17, 2020

(54) PHOTOCATALYTIC METHOD FOR DISINFECTION OF INTERIOR SURFACES

(71) Applicants: Räzvan Cätälin BUCURESTEANU, Bucharest (IT); Gheorghe-Mihai STÃRUS, Vienna (AT)

(72) Inventor: Räzvan Cätälin BUCURESTEANU, Bucharest (RO)

(21) Appl. No.: 16/754,450

(22) PCT Filed: Oct. 8, 2018
(86) PCT No.: PCT/RO2018/000018

§ 371 Date: Apr. 8, 2020

(30) Foreign Application Priority Data
Oct. 9, 2017 (RO) A/2017 00801

(51) Int. Cl. C09D 5/14; A61L 2/08; C09D 125/08; C09D 175/04

(52) U.S. Cl. CPC C09D 5/14; A61L 2/084; C09D 175/04; C09D 125/08; A61L 2/088

(57) ABSTRACT

The present invention refers to a Photocatalytic method for disinfection of interior surfaces and Composition of washable biocide paint with photocatalytic properties used to realize the antimicrobial and antifungal protection and disinfection of interior surfaces in medical practices, hospitals, schools, food industry, in areas where is a danger of occurrence and spread of microbial germs, composition which is applied directly on interior walls by painting. The composition in accordance to this invention is based on acrylic-styrene resins in which was dispersed as photosensitive biocide agent, particles of photosensitized metal oxide semiconductor of type anatase Ti02 or ZnO, oxides that are doped with transition metals like Ag, mainly, or Au, or with Cu, Ni, Fe, Cr, Co, Mn, and the biocide properties are activated through a photocatalytic activation Method by irradiating the composition with photons in the visible light spectrum, with wavelengths between 450 nm and 500 nm, which are characteristic for the activation of the dopant from the photosensitized semiconductor of type anatase Ti02 or ZnO, determining the apparition of reactive Oxygen singlet species ROS (type O₂ ¹Ag or O₂ ¹.r,g+), species which have biocide and disinfecting action. Thus is obtained a disinfection process of the interior surfaces, which is controlled, adjustable, by the intensity of light function of disinfection necessity, reproducible, without the variation influence of external luminous factors.

The present invention refers to a Photocatalytic method for disinfection of interior surfaces and composition of biocide washable paint with photocatalytic properties, composition based on aqueous acrylic-styrene resins, in which was dispersed as photosensitive biocide agent, metal oxides semiconductor particles such as anatase Ti02 or ZnO, which are doped with transition metals such as Ag mainly, or Au, or with Cu, Ni, Fe, Cr, Co, Mn and a photocatalytic method of activation of the biocide component and of the disinfecting properties of the composition using visible light quanta between 450 nm and 500 nm, function of used doping agent, to realize the antimicrobial and antifungal protection and disinfection of interior surfaces, in medical practices, hospitals, schools, food industry, generally in areas where is a danger of occurrence and spread of microbial germs. The composition to which the patent refers is applied on the interior walls of the rooms by painting all kinds of interior surfaces made of masonry, concrete, plaster or any other mineral material based surface including cellular concrete, bricks, or plasterboard, gypsum, as a washable biocide protective paint which contains photosensitive pigment of anatase Ti02, or ZnO, doped with transition metals as Ag, generally, or Au, or with Cu, Ni, Fe, Cr, Co, Mn. Ensuring of the disinfection and biocide effect of the composition so applied on the wall, is realized via a photocatalytic activation method of the photosensitive agent from the biocide composition using permanent or intermittent irradiation as per need, as well as the source of the luminous irradiation of the respective surfaces with quanta of light with a wavelength between 450 nm and 500 nm, the light being emitted by interior lighting lamps. The wavelengths emitted in the spectrum 450 nm and 500 nm are specific wavelengths to which is activated the Ti02 or ZnO doped with transition metals like Ag, generally, but also Au, Cu, Ni, Fe, Cr, Co, Mn. When Ti02 is excited by the luminous energy equal to or greater than the width of the forbidden band, photodynamic reactions are generated, at the photosensitizer agent level, having as result the formation of the reactive species of the ROS singlet oxygen with bactericidal action against pathogenic germs. By ROS are understood the species of reactive oxygen radicals which appear as a result of the electron transfer from the semiconductor substrate to the free oxygen molecules, the ROS species being much more reactive to organic molecules than of the molecular oxygen itself. In this manner is obtained a disinfection process of the interior surfaces, which is controlled, adjustable, by the intensity of light function of disinfection necessity, reproducible and without the variation influence of external luminous factors.

There are known different compositions of washable biocide paint which contain as biocide agent either active substances of type benzisothiazol-3(2H)-one or silver particles or colloidal silver or mixture of benzisothiazol-3(2H)-one with silver ions. The disadvantage of such compositions is that the benzisothiazol-3(2H)-one creates contact allergies and have a limited action in time as well as upon microbial germs that develop resistance. Silver, although is a much better biocide than benzisothiazol-3(2H)-one, has a limited action, the antiseptic effect depending on the concentration and the shape of the ions within the paint.

It is known that semiconductor metallic oxides of the type anatase Ti02 or ZnO have a photosensitive role in the photocatalytic reactions. It is also known the effect and the action mode in the photodynamic therapy of photosensitizers, based on photochemical reactions, triggered by the interaction of a substance photosensitive at light with a certain wavelength, form the reactive species of oxygen singlet ROS (type 02 1 g or 02 1 Lg+). The disinfecting action of the metal oxide semiconductor photosensitizers such as anatase Ti02 or ZnO is performed on the basis of a photocatalytic mechanism, triggered by the interaction of the photosensitizer agent, which contains anatase Tio2 or ZnO, with light having a certain wavelength, after which appear the reactive species of oxygen-oxygen singlet ROS, having a determining role in the destruction of the microorganisms, thus conferring these substances a biocide and antifungal role.

One of the first examples of application of the semiconductor photocatalysis as disinfection method was the work of Matsunaga and contributors [T. Matsunaga, R. Tomoda, T. Nakajima, N. Nakamura, T. Komine, f-Q1 Appl. Environ. Microbiol. 54 (1988) page 1330]. They succeeded to demonstrate that Ti02 particles irradiated in the ultraviolet spectrum were efficient in the photo-destruction of bacteria such as Lactobacillus acidophilus, Saccharomyces cerevisiae and Escherichia coli and that the destruction was associated with the reduction of intracellular CoA level through photo-oxidation. In another study Cushnie and contributors [T. P. T. Cushnie, P. K. J. Robertson, S. Officer, P. M Pollard, R. Prabhu, C. McCullagh, J. M. C. Robertson Photobactericidal effects of Ti02 thin films at low temperatures—A preliminary study J. Photoch. Photobio. A, 216 (2010), pp. 290-294] demonstrated and evaluated the very good antibacterial efficiency of the anatase Ti02, activated by UV on Staphylococcus aureus including experiments done in low temperature. In another study U. Joost and colaborators [U. Joost, K. Juganson, M. Visnapuu, M. Mortimer, A. Kahru, E. Nommiste, U. Joost, V. Kisand, A. lvask, Photocatalytic antibacterial activity of nano-Ti02 (anatase)-based thin films: effects on Escherichia coli cells and fatty acids, Journal of Photochemistry and Photobiology B: Biology (2014)] demonstrated the distinguished efficacy of Ti02 photocatalytic activated by UV as bactericidal agent upon Escherichia coli. In another study from 2015 Fagan shows that simple Ti02 or doped with Ag or Au, Cu, Ni has excellent photocatalytic bactericidal properties and explains the mechanism of photocatalytic biocide action of Ti02 [Fagan, R. et al., (2015) A review of solar and visible light active Ti02 photocatalysis for treating bacteria, cyanotoxins and contaminants of emerging concern, Materials Science in Semiconductor Processing, vol. 42, pp. 2-14]

In the patent: DE202015000762U is described an universal lighting panel for lamps covered with Ti02 and which has an odor neutralizing function and a hygienic function. In the patent W02011/113692A1 is described a procedure for production of plastic panels covered with photocatalytic Ti02 with biocide properties. In the patent US 20140205546A1 is described the realization of a polymeric thin film with Ti02 doped with silver.

The major disadvantage of such applications for photocatalytic disinfection is that they use for photocatalytic activation either the UV radiation—which is dangerous for humans—or the natural radiation given by solar radiation and due to this fact has a small quantum yield, taking into account that solar radiation contains less than 5% photons with specific wavelength which activates the photosensitizers with Ti02. That is why the photo-disinfection with Ti02 has applications, which can be tolerated in long contact periods and where abundant sunlight exists, but the quantum yields, as efficiency of disinfection process, registers fluctuations given by the intensity of solar radiation.

Another major disadvantage of such applications is given by the fact that none of these applications does not realize a coverage of the total surface of interior walls of the rooms.

The present invention Photocatalytic method for disinfection of interior surfaces and Composition of washable biocide paint with photocatalytic properties solves these technical issues through realization of a photosensitive biocide composition applicable as washable paint on the surface of interior walls and which contains biocide pigments with biocide photocatalytic action based on anatase Ti02 or ZnO doped with transition metals of type Ag, or Au, Cu, Ni, Fe, Cr, Co, Mn and the combining action of the composition with a photocatalytic method for the activation of the photosensitizer particles from the composition by illumination of the walls covered by the described composition by light in visible spectrum and having wavelengths between 450 nm and 500 nm emitted by illumination lamps in the respective spaces, light which initiates the photocatalytic disinfection process through the activation of the pigment anatase Ti02 or ZnO, doped with Ag or Au, Cu, Ni, Fe, Cr, Co, Mn.

The first technical problem solved by the present invention consist m obtaining a composition of washable paint type with a powerful antibacterial and anti-allergen action with high covering properties, which allows a total covering with a film protecting antibacterial material of the interior walls from hospitals, medical practices, schools, food industry spaces or other type of spaces which represents danger of occurrence and spread of infections by microbial germs or nosocomial infections, composition in which was uniformly dispersed in its mass a photosensitizer based on anatase Ti02 or ZnO, doped with transition metals, especially with Ag, but can be also used as dopants Au, Cu, Ni, Fe, Cr, Co, or Mn, photosensitizer which is activated by photons with wavelengths of the visible spectrum, especially wavelengths between 450 nm and 500 nm.

The second technical problem solved by the present invention is the method of photocatalytic activation of the photosensitizer from the composition, by irradiation of the applied composition on interior walls with photons emitted from the illumination lamps of interior spaces, lamps which contain also irradiation sources which emit continuous, pulsed or intermittent light in the spectrum between 450 nm-500 nm, lamps fixed to the ceiling of the rooms, or LED strips mounted on the walls of the rooms or mobile lamps that illuminate according to the disinfection requirements.

According the invention Photocatalytic method for disinfection of interior surfaces and Composition of washable biocide paint with photocatalytic properties the applied composition by painting the interior walls of the rooms is based on acrylic-styrene resin between 5-15 parts, polyurethane resin between 5-15 parts, propylene glycol up to 10 parts, water between 5 and 35 parts, white pigment between 5 and 20 parts, filler material up to 50 parts, rheology additives up to 10 parts, pH balancer, dispersing agent 0.2-2 parts, anti-foaming agent 0.2-2 parts, surfactant between 0.2-1 parts, cellulose hardener between 0.2-2 parts to which is added in the composition between 10 and 20 parts pigment as a photocatalytic biocidal agent based on anatase Ti02 doped with Ag ions between 0.7% and 1.5% from the mass of photosensitizer agent, parts express by weight. It can be used as photosensitizer pigment, instead of metal oxide anatase Ti02 also ZnO doped with Ag or it can be doped with other transition metals of type Au, Cu, Ni, Fe, Cr, Co or Mn. The disinfection process is triggered by the biocide composition applied on the interior walls of the rooms under the form of washable paint by its activation with a photocatalytic method of irradiation of the walls covered with this washable paint using emitted photons by the illumination lamps of the interior spaces, lamps which contain also irradiation sources which emit continuous, pulsed or intermittent light also between also in the spectrum of 450 nm-500 nm, lamps fixed on the ceilings as well as led strips applied on the walls of rooms, or mobile lamps which illuminate function of disinfection necessity, initiating the photocatalytic disinfection process of the pigment anatase Ti02 pigment or the pigment ZnO, doped with Ag or doped with Au, Cu, Ni, Fe, Cr, Co or Mn, pigments from the composition of washable biocide paint. This photocatalytic method of activation of the photosensitive particles from the biocide composition ensure a duration and an intensity of illumination according to the disinfection necessities. Following the application of this photocatalytic method by activation of photosensitizing particles from the photocatalytic biocidal composition, the reactive species of singlet oxygen ROS (type 02 1 g or 02 1 Lg+) appear, species which have a biocide and disinfectant action, having a determining role in microorganism destruction and confer to the photocatalytic composition an antibacterial and antifungal function. Thus is obtained a disinfection process of interior surfaces controlled, adjustable by luminous intensity, function of the disinfection necessities, reproducible, and without being influenced by the variation of external luminous factors.

The photocatalytic method for disinfection of interior surfaces and the composition of biocide washable paint with photocatalytic properties has a powerful biocidal action by cumulating the biocide action of Ag ions with the photocatalytic biocide effect of the photosensitizer type anatase Ti02 or of the photosensitizer metal oxide semiconductor ZnO. By doping these pigments with Ag ions (or other transition metals such as Au, Cu, Ni, Fe, Cr, Co or Mn) the activation spectrum of the photosensitizer anatase Ti02 as well as of metal oxide semiconductor ZnO, is shifted towards wavelengths of visible spectrum, thus enabling a permanent activation method without danger for humans, eliminating the activation with waves from UV spectrum, dangerous for humans.

By application of the invention Photocatalytic method for disinfection of interior surfaces and the Composition of biocide washable paint with photocatalytic properties the following advantages are obtained:
Realization of a total coverage with film protecting antibacterial material of interior walls, eliminating the transmission of nosocomial infections
Cumulating the biocide effects of the Ag ions with the photocatalytic biocide effect of the photosensitizer type anatase Ti02 or of the metal oxide semiconductor ZnO
By doping with Ag ions (or other transition metals such as Au, Cu, Ni, Fe, Cr, Co or Mn) the activation spectrum of the photosensitizer anatase Ti02 a or of the metal oxide semiconductor ZnO is shifted towards wavelengths of visible spectrum
This eliminates the need to use the photosensitizer, which is activated by wavelengths from UV light, dangerous for humans
Thus was realized, an activation method of the photosensitizer which is not dangerous for humans, thus enabling its permanent and total activation against microbial germs
Does not show allergic phenomena, being an ecological product
Easy manufacturing process of the composition, because the photosensitizers used are fully compatible with the aqueous resins used in the composition
Resistance to yellowing
High whiteness degree, ensures breathing of the wall

### REALIZATION EXAMPLE

The composition is obtained using a Cowles type disperser, introducing 15 liters of water, 10 kg propylene glycol, 10 kg polyurethane resin, 25 kg filler agent. The composition is dispersed 20 minutes and in increments are added 15 kg of anatase Ti02 pigment—biocide anatase photocatalytic doped agent with Ag ions between 0.7% and 1.5%. After 20 minutes dispersion is added, 1 kg of dispersing agent, 1 kg of anti-foaming agent, 2 kg of cellulose hardener, 15 kg of acrylic-styrene resin, pH balancer and is filled with water until 100 kg. It is stirred and shaken continuously until a dispersion mass is obtained as a homogeneous viscous liquid.

1.-9. (canceled)

10. (canceled)

11. (canceled)

12. A composition of washable biocide paint which contains biocide pigments with biocide photocatalytic action based on a photosensitizer of TiO2 anatase or Zn doped with transition metals of Ag, Au, Cu, Ni, Fe, Cr, Co and Mn.

13. The composition according to claim 12 further comprising:
acrylic-styrene resin between 5 to 15 parts by weight,
polyurethane resin between 5 to 15 parts by weight,
propylene glycol up to 10 parts by weight,
water between 15 to 35 parts by weight,
white pigment between 5 to 20 parts by weight,
filler material up to 50 parts by weight,
rheology additives up to 10 parts by weight,
pH balancer,
cellulose hardener between 0.2 to 2 parts by weight
wherein parts by weight are based to the total weight of the composition,
and comprising between 10 to 20 parts by weight of the biocide pigment.

14. The composition according to claim 13 wherein the TiO2 anatase or ZnO is doped with between 0.7% and 1.5% of transitional metals of Ag, Au, Cu, Ni, Fe, Cr, Co or Mn from the photosensitizer mass.

15. The composition according to claim 13 comprising:
acrylic-styrene resin 15 parts by weight,
polyurethane resin 10 parts by weight,
propylene glycol 10 parts by weight,
white pigment between 5 to 20 parts by weight,
filler material 25 parts by weight,
rheology additives up to 10 parts by weight,
pH balancer,
dispersing agent between 0.2 to 2 parts by weight,
anti-foaming agent between 0.2 to 2 parts by weight,
surfactant between 0.2 to 1 parts by weight,
cellulose hardener between 0.2 to 2 parts by weight and water up to 100 parts
wherein parts by weight are based to the total weight of the composition
and comprising 15 parts by weight parts by weight of the biocide pigment.

16. The composition according to claim 15 wherein the TiO2 anatase or ZnO is doped with between 0.7% and 1.5% of transitional metals of Ag, Au, Cu, Ni, Fe, Cr, Co or Mn from the photosensitizer mass.

17. A photocatalytic method for disinfection of interior surfaces comprising
photocatalytic activation generated by irradiating the surfaces painted with the composition of claim 12 with irradiation sources which emit continues, pulsed or intermittent light in the spectrum between 450 nm and 500 nm,
wherein the light is emitted by the interior lighting lamps that provides a continuous disinfection of the interior surfaces controlled, adjustable by the light intensity according to the needs of disinfection, reproducible and without the influence of the external light factors,
and wherein the light emitted by the lighting lamps assures both the photocatalytic activation of composition of claim 12 and the necessary light for conducting the activity in good conditions in the respective premises.

18. A photocatalytic method for disinfection of interior surfaces comprising
photocatalytic activation generated by irradiating the surfaces painted with the composition of claim 13 with irradiation sources which emit continues, pulsed or intermittent light in the spectrum between 450 nm and 500 nm,
wherein the light is emitted by the interior lighting lamps that provides a continuous disinfection of the interior surfaces controlled, adjustable by the light intensity according to the needs of disinfection, reproducible and without the influence of the external light factors,
and wherein the light emitted by the lighting lamps assures both the photocatalytic activation of composition of claim 13 and the necessary light for conducting the activity in good conditions in the respective premises.

19. A photocatalytic method for disinfection of interior surfaces comprising
photocatalytic activation generated by irradiating the surfaces painted with the composition of claim 14 with irradiation sources which emit continues, pulsed or intermittent light in the spectrum between 450 nm and 500 nm,
wherein the light is emitted by the interior lighting lamps that provides a continuous disinfection of the interior surfaces controlled, adjustable by the light intensity according to the needs of disinfection, reproducible and without the influence of the external light factors,
and wherein the light emitted by the lighting lamps assures both the photocatalytic activation of composition of claim 14 and the necessary light for conducting the activity in good conditions in the respective premises.

20. A photocatalytic method for disinfection of interior surfaces comprising
photocatalytic activation generated by irradiating the surfaces painted with the composition of claim 15 with irradiation sources which emit continues, pulsed or intermittent light in the spectrum between 450 nm and 500 nm,
wherein the light is emitted by the interior lighting lamps that provides a continuous disinfection of the interior surfaces controlled, adjustable by the light intensity according to the needs of disinfection, reproducible and without the influence of the external light factors,
and wherein the light emitted by the lighting lamps assures both the photocatalytic activation of composition of claim 15 and the necessary light for conducting the activity in good conditions in the respective premises.

21. A photocatalytic method for disinfection of interior surfaces comprising
photocatalytic activation generated by irradiating the surfaces painted with the composition of claim 16 with irradiation sources which emit continues, pulsed or intermittent light in the spectrum between 450 nm and 500 nm,
wherein the light is emitted by the interior lighting lamps that provides a continuous disinfection of the interior surfaces controlled, adjustable by the light intensity according to the needs of disinfection, reproducible and without the influence of the external light factors,
and wherein the light emitted by the lighting lamps assures both the photocatalytic activation of composition of claim 16 and the necessary light for conducting the activity in good conditions in the respective premises.
